(19) United States
(12) Patent Application Publication
(10) Pub. No.: US 2023/0149735 A1
(43) Pub. Date: May 18, 2023

(54) ANTI-INFECTIVE LIGHT RADIATION METHODS AND DEVICES

(71) Applicant: Mike Miskin, Sleepy Hollow, IL (US)

(72) Inventor: Mike Miskin, Sleepy Hollow, IL (US)

(21) Appl. No.: 17/920,619

(22) PCT Filed: Apr. 23, 2021
(86) PCT No.: PCT/US2021/028863

§ 371 Date: Oct. 21, 2022

Related U.S. Application Data
(60) Provisional application No. 63/101,293, filed on Apr. 23, 2020.

(51) Int. Cl. A61N 5/06

(52) U.S. Cl. CPC A61N 5/0624; A61N 2005/0651; A61N 2005/0659; A61N 2005/0661; A61N 2005/0663

(57) ABSTRACT

Anti-infective light radiation methods and devices are disclosed. In an example, an anti-infective radiation device includes a lighting device configured to provide an output energy of at least one antimicrobial electromagnetic radiation wavelength(s) within a range of 350 nm - 450 nm. The lighting device is configured to be directed towards an exterior of a living species and/or integrated and/or placed within an interior of a living species. The example lighting device projects one or more sufficient levels of the electromagnetic wavelength radiation directly onto and/or through one or more layers of living species tissue so that the electromagnetic wavelength radiation reaches near or directly onto unwanted infectious living cells or organisms. The antimicrobial electromagnetic radiation damages or kills unwanted infectious cells or organisms on or within the living species.

### TECHNICAL FIELD

[Para 1] The present invention generally relates to delivering visible light and/or infrared light radiation to living species to reduce and/or kill infections and/or infectious organisms on or within living species. Specifically, the present invention relates to methods and devices for delivering and projecting antimicrobial and/or infrared “IR” lighting radiation (anti-infective light radiation or “ALR”) for eliminating infections internal to living species including but not limited to humans, animals, mammals and other living species.

### BACKGROUND

[Para 2] Electromagnetic radiation has been used to treat cancer and is known to be dangerous. In addition to treating cancer, radiation oncologists may use ionizing radiation to treat benign tumours that are unresectable (unable to be removed by surgery), such as certain types of tumours occurring in the brain (e.g., craniopharyngiomas and acoustic neuromas). Until the significant long-term consequences of ionizing radiation were recognized, radiation therapy was sometimes used for conditions such as acne, tinea capitis (ringworm of the scalp and nails), and lymph node enlargement, but those uses were abandoned following the discovery of ionizing radiation injury. Early radiation therapy machines produced X-rays that were in the orthovoltage range (between about 140 and 400 kilovolts). That treatment caused serious and often intolerable skin bums. Modem radiation therapy machines produce beams that are in the high-energy megavoltage range (more than 1,000 kilovolts), which allows the beam to penetrate tissues and treat deep-seated tumours. The dose to the skin, however, is lower than with orthovoltage treatment.

[Para 3] The majority of modern radiation therapy treatments are external beam teletherapy, or long-distance therapy (sometimes also called external beam radiotherapy). External beam machines produce ionizing radiation either by radioactive decay of a nuclide, most commonly cobalt-60, or through the acceleration of electrons or other charged particles, such as protons. Most radiation therapy treatments use irradiation generated by linear accelerators, which impart a series of relatively small increases in energy to particles such as protons, carbon ions, or neutrons. The accelerated particles bombard a target, which then produces the therapeutic beam of radiation. The energy of the beam is determined by the energy of the accelerated particles. Two commonly used approaches to external beam teletherapy are intensity-modulated radiation therapy (IMRT) and particle beam therapy.

[Para 4] Another technique used for the delivery of radiation is known as brachytherapy. In that form of therapy, radiation is implanted directly into a tumour or tumour-bearing tissue. The encapsulated radioactive sources are inserted into the tumour via catheters or needles. A catheter can be placed into a tumour bed after tumour resection, whereas a needle can be inserted into the affected tissue directly or into the body cavity housing the affected tissue. In both cases, radioactive sources are carefully threaded into the delivery device. Brachytherapy is valuable in particular because it can deliver a high dose of radiation to the tumour tissue or tumour bed while sparing the surrounding healthy tissue.

[Para 5] It has been known for several decades that the use of light can give a positive therapeutic effect in the treatment of a wide spectrum of diseases. In the 1960’s the use of narrow wavelength light was investigated in vivo/in vitro experiments. It was found that light of wavelength greater than 440 nm did not work. Further investigations were carried out with light having a wavelength of from 300 to 350 nm (UV light) but it was found that infection was exacerbated/promoted rather than ameliorated/eliminated. Some attempts have been made to treat individuals affected with the herpes virus by treatment with light of the wavelength 660 nm, as described in U.S. Pat. No. 5,500,009.

[Para 6] Additionally, it is known from the prior art to use a laser to produce coherent radiation and to focus it on the area to be treated. Nd YAG laser treatment at a fundamental wavelength of 1064 nm is associated with decreased pain, scarring and improved healing (U.S. Pat. No. 5,445,146). Additionally it has been reported that diodes emitting light at the red wavelength, 940±25 nm can be used to treat a range of essentially musculoskeletal ailments (U.S. Pat. No. 5,259,380). However there is no indication that light of a wavelength above this would be of any therapeutic use.

[Para 7] It has now been surprisingly established that low intensity electromagnetic radiation of small bandwidth is effective in the treatment of infectious diseases, inflammatory-type diseases and other conditions, including the alleviation of pain. It is postulated that the way in which the electromagnetic radiation effects its action is by way of energy transmission through cellular components/organelles.

[Para 8] A water molecule that has a range of electromagnetic radiation wavelengths passed through it will produce several transmission peaks. These transmission peaks can be associated with the preferred therapeutic electromagnetic radiation wavelengths and/or ranges used in the invention and thus implies there may be a role for the water molecule in the general mechanism of action.

[Para 9] Ultraviolet (UV) light has been used to reduce and/or kill unwanted microorganisms and/or bacteria. Ultraviolet (UV) radiation is electromagnetic radiation with a wavelength (100-400 nm) shorter than that of visible light (400-700 nm), but longer than x-rays (<100 nm). UV irradiation is divided into four distinct spectral areas including vacuum UV (100-200 nm), UVC (200-280 nm), UVB (280-315 nm) and UVA (315-400 nm). The mechanism of UVC inactivation of microorganisms is to damage the genetic material in the nucleus of the cell or nucleic acids in the virus. The UVC spectrum, especially the range of 250-270 nm, is strongly absorbed by the nucleic acids of a microorganism and, therefore, is the most lethal range of wavelengths for microorganisms. This range, with 262 nm being the peak germicidal wavelength, is known as the germicidal spectrum. The light-induced damage to the DNA and RNA of a microorganism often results from the dimerization of pyrimidine molecules. In particular, thymine (which is only found only in DNA) produces cyclobutane dimers. When thymine molecules are dimerized, it becomes very difficult for the nucleic acids to replicate and if replication does occur it often produces a defect that prevents the microorganism from being viable.

[Para 10] Although it has been known for the last 100 years that UVC irradiation is highly germicidal, the use of UVC irradiation for prevention and treatment of infections is still in the very early stages of development. Most of the studies are confined to in vitro and ex vivo levels, while in vivo animal studies and clinical studies are much rarer. Studies that have examined UVC inactivation of antibiotic-resistant bacteria have found them to be as equally susceptible as their naive counterparts. Within the UVC range, 254 nm is easily produced from a mercury low-pressure vapor lamp, or more recently light emitting diode “LED” technology and has been shown to be close to the 262 nm optimal wavelength for germicidal action. Because the delivery of any UV light, including UVC irradiation to living tissue is a localized process and introduces added risk of damaging and/or destroying good, healthy living cells similar to that of microwave , UVC for infectious diseases is likely to be applied exclusively to localized infections more often as a last resort solution.

[Para 11] The Infrared “IR” radiation energy spectrum falls within the range of approximately 700 nm - 1 mm often broken into categories and referred to as one of either Near Infrared “NIR”, Mid Infrared “MIR” or Far Infrared “FIR” energy. One or more of these IR energy are often used in various types of light therapy including but not limited to dermatology, hair growth and saunas.

[Para 12] NIR energy is cooler than the others so it may be much easier for some people to handle. It has a detoxing effect on the body that includes the following benefits: Heals wounds due to the regeneration of cells, especially in your skin, muscles, and tendons. Anti-aging also due to regeneration of cells and its antioxidant properties. Improves oxygen delivery to your cells. Improves your overall health because it enables the body to perform metabolic processes better.

[Para 13] MIR energy reaches deeper into your body providing some other benefits: Better blood circulation. Reduced pain and inflammation due to increased blood circulation and oxygen delivery. Quicker recovery from injury. Weight loss.

[Para 14] FIR energy reaches the deepest and heats up your core. It has the following benefits: Detoxification due to producing sweat that comes from deep within removing the toxins. Relaxation due to the heat penetrating deeply. Lower blood pressure because the heat allows your arteries to dilate.

[Para 15] LED lighting devices have been developed to emit near UV and/or visible light that also kills bacteria and is safer on living species cells but require more time to kill microorganisms than conventional UV light sources at taught by Lalicki et al. in US Pat. numbers 9927097 and 10357582. These devices emit a majority of light/peak of light within the 380-420 nm wavelength range rather than wavelengths within the conventional range of visible light at approximately 450-495 nm, which would be perceived as blue and then coated and/or covered with a phosphor to enable the blue to be converted to a more natural white light.

[Para 16] Light in the 380-420 nm wavelength is capable of killing or deactivating microorganisms such as but not limited to Gram positive bacteria. Gram negative bacteria, bacterial endospores, and yeast and filamentous fungi. Some Gram positive bacteria that can be killed or deactivated include Staphylococcus aureus (incl. MRSA), Clostridium perfringens, Clostridium difficile, Enterococcus faecalis, Staphylococcus epidermidis, Staphyloccocus hyicus, Streptococcus pyogenes, Listeria monocytogenes, Bacillus cereus, and Mycobacterium terrae. Some, Gram negative bacteria include Acinetobacter baumannii, Pseudomonas aeruginosa, Klebsiella pneumoniae, Proteus vulgaris, Escherichia coli, Salmonella enteritidis, Shigella sonnei, and Serratia spp. Some bacterial endospores include Bacillus cereus and Clostridium difficile. Some yeast and filamentous fungi include Asrpergillus niger, Candida albicans, and Saccharomyces cerevisiae. Light in the 380-420 nm wavelength has been effective against every type of bacteria tested, although it takes different amounts of time or dosages dependent on species Based on known results it is expected to be effective against all gram-negative and gram-positive bacteria to some extent over a period of time. It can also be effective against many varieties of fungi, although these will take longer to show an effect.

[Para 17] LED lighting systems that use 405 nm and/or in the range of 380 - 420 nm antimicrobial properties have recently been tested and implemented into products available in the market for general lighting purposes. These devices and/or systems use wavelengths between 380 - 420 nm, 405 nm for example and coat the 405 nm LEDs with phosphor so that both white light and antimicrobial light is delivered from the lighting system. The rate at which these lighting systems kill unwanted microbes varies based on the level of light and or lux output being projected onto a specific surface. Although these LED lights take longer to kill microbes compared to the lower UV wavelength alternatives, they are safer for people. An important variable in testing the efficacy of 405 nm light is the lux level of lights being used. Lux is the standard unit of measure of illuminance and luminous emittance, measuring the perceived power of light per unit area It is equal to one lumen per square meter and is used as a measure of brightness, as perceived by the human eye, of light that hits or passes through a surface, and similarly would be in the case of the proposed invention described herein, onto and/or through living tissue to reduce and or eliminate microbial infections.

[Para 18] LEDs are also available in various IR wavelengths and can likely be manufactured to offer any wavelength in the range of 700 nm ― 1 mm in the IR spectrum. The benefit of LEDs is that they can be manufactured to deliver very specific wavelengths.

[Para 19] LEDs are semiconductor devices that produce light when a current is supplied to them. LEDs are intrinsically DC devices that only pass current in one polarity and have historically been powered and/or driven with constant current or constant voltage DC power supplies however recently LEDs have also been driven with AC voltages and/or rectified high voltage AC. LEDs can therefore be driven with AC and/or DC using complex, or simple power supplies and/or drivers, as well as with batteries as they have been in flash lights and other battery backup lighting systems. With the recent high growth and use of LED technology, LEDs have more recently often been designed into humancentric lighting systems, plant growth systems, dermatology lighting systems and are more often being tested and developed for medical applications.

[Para 20] The epitaxial growth process of LEDs is reasonably precise and allows LED chip manufacturers to provide many various wavelength options. LED chips can be packaged with or without phosphors based on the designer light output color (ie. red, green, blue, violet) and/or visible or non-visible wavelength. Phosphors and/or nano-crystals can be used to convert the original output color and/or wavelength of a LED chip to a white and/or near white light color temperature. White light color temperatures are often measured in Kelvins “K” and can range from 1500 K (in the red and/or candlelight range) to 7500 K (more blue Ultra Daylight) range. Wavelengths, colors and/or color temperatures of light can be combined, mixed and/or modulated to produce net resultant outputs of different wavelengths, colors and/or color temperatures of light. This can be done with various types of software and/or hardware including but not limited to drivers, microprocessors, controllers, modulation methods, pulsed outputs and other such methods and/or devices that could be integrated in various types of lighting systems, including but not limited to the proposed antimicrobial lighting devices for eliminating microbial infections in living species and/or living tissue as described herein.

[Para 21] LED chips are most often packaged with similar types of wavelengths if more than one chip is integrated in a single package, assembly or substrate such as blue/blue, red/red and so on. However Red, Green, Blue “RGB” is also a common LED package. The RGB LEDs and/or lighting systems are often used in LEDs signs, displays, theater lighting and other lighting systems where color changing is a requirement. Some LED packages and/or assemblies have included Blue and Red chips or LED packages mixed together to increase the quality of the light and/or color rendering index “CRI”. An alternative to using red LEDs is to just use blue and adjust the phosphor coating on the blue LED chips so that the white light output from the LED package and/or assembly has increased red color to it.

[Para 22] In recent months, the world has been affected with a global pandemic resulting in a significant number of rapidly increasing infections and loss of life as a result of the Coronavirus, more specifically COVID-19. COVID-19 is another dangerous respiratory infection that can lead to pneumonia and death similar to SARS and MERS. Doctors and scientists around the world are working fast to develop treatments, vaccines, equipment and more to help combat the global pandemic. Some of the proposed solutions being used include already approved medications such as hydroxychloroquine however many others are not yet tested and can have negative effects on the human living species they’re being designed for.

[Para 23] Past viral pandemics and now COVID-19 have proven to put the worlds populations and economies at risk. Unfortunately, it’s likely that this can occur again one day on the future. Other infections, for example kidney, diabetic limb and more occur on a regular bases and often lead to undesirable negative results. New solutions are needed for current and future microbial infectious diseases. It is contemplated to use a non-pharmaceutical technology based solution using light to kill microbial infections within living species and/or tissue.

[Para 24] Therefore, it would be advantageous to provide antimicrobial lighting devices and methods for eliminating microbial infections in living species including but not limited to humans, animals, mammals and other living species.

[Para 25] It would also be advantageous to design a circuits, devices and lighting systems that use LEDs, OLEDs, Laser, Halogen, Xenon, Mercury Vapor or any other lighting technology that can be used to achieve and deliver the desired results and performance needed to reduce and/or eliminate microbial infections in specific areas of living species and/or living tissue.

[Para 26] The present invention is provided to solve these and other issues.

### SUMMARY

[Para 27] The present invention relates to methods and devices for delivering and projecting antimicrobial and/or infrared “IR” lighting radiation (anti-infective light radiation or “ALR”) for eliminating infections internal to living species including but not limited to humans, animals, mammals and other living species. The present invention uses lighting devices, that from the exterior of a living species and_{/}or when integrated or placed within the interior of a living species, project sufficient levels of visible light and/or IR radiation directly onto and/or through one or more layers of living tissue so that the visible light and/or IR radiation energy reaches infectious organisms.

[Para 28] The present invention may also use antimicrobial lighting devices that produce one or a combination and/or group of electromagnetic radiation energy wavelengths in the range of 350 ― 450 nm, and more specifically 380 ― 420 nm, and/or use Red and/or infrared electromagnetic radiation (IR) lighting and/or devices that produce one or a combination and/or group of electromagnetic radiation energy wavelengths in the range of 625 ―1200 nm. In some instances, the present invention individually uses the IR radiation and/or wavelengths to increase heat onto and/or near the infectious organisms. The invention may simultaneously apply and/or project the antimicrobial lighting and the Red and/or IR lighting radiation and/or wavelengths onto and/or near the infections to reduce and/or kill invading and/or unwanted infectious organisms, on and/or within a living species.

[Para 29] The disclosed invention is directed to “anti-infective lighting radiation “ALR” methods and devices “ALRMD” for eliminating infections in living species”. ALRMD can include, but is not limited to, using light emitting diodes, fluorescent, halogen, or any other light sources that can emit any single wavelength or combination of wavelengths in the range of visible and/or non-visible light spectrums such as wavelengths including but not limited to UV, near UV, and/or other visible and/or non-visible wavelengths at various levels of constant, pulsed and/or modulated energy intensities that may be used to harm, destroy and/or prevent infectious organisms from multiplying on environmental surfaces, and more specifically as described herein, on or within living species.

[Para 30] Such ALRMD may be powered with AC mains voltage sources, low voltage power supplies, batteries and/or any form of power source sufficient to power a specific ALRMD and/or system. The ALRMD may provide different levels of brightness and/or intensities of output wavelengths of visible and/or non-visible light by switching or controlling the wavelengths in response to one or more control devices and/or methods including but not limited to sensors, controllers, microprocessors, biofeedback, integrated circuits and/or other wavelength management and/or control circuitry or user or operator of the ALRMD. The sensors can include but not be limited to sensors capable of sensing one or more of temperature, electrical signals, humidity, blood, microorganisms, organisms, biofeedback, oxygen, enzymes, fluids and/or minerals.

[Para 31] Such ALRMD may also include circuitry to allow for controlling and/or programming the output wavelengths for timing, duration, which wavelengths to be used and when as well as the intensity levels of such wavelengths. The ALRMD may include wired and/or wireless communication and/or control, by medical personnel and/or other practitioners, operators and/or users of the ALRMD.

[Para 32] According to one aspect of the invention, the present invention provides methods and devices including but not limited to Anti-Infective Light Radiation and/or antimicrobial lighting devices for eliminating microbial infections in living species and/or living tissue. The present invention specifically relates to Anti-Infective Light Radiation “AILR” methods and devices “AILR-MD” for eliminating microbial, parasitic, cancerous and other infections on the exterior and/or interior of living species including but not limited to humans, animals, mammals and other living species by:
a.) providing and using lighting devices and/or systems, that from the exterior of a living species and/or when integrated or placed within the interior of a living species, will project and/or radiate sufficient levels of electromagnetic radiation of light and/or IR energy directly onto and/or through one or more layers of living tissue so that the light and/or IR energy reaches unwanted infectious organisms, with such devices and methods including but not being limited to:
b.) providing and using antimicrobial lighting devices that produce one or a combination and/or group of electromagnetic radiation wavelengths in the range of 350-450 nm, and more specifically 380-420 nm, and/or using Red and/or infrared (IR) lighting and/or devices that produce one or a combination and/or group of electromagnetic radiation wavelengths in the range of 625-1200 nm, and;
c.) individually using the IR electromagnetic radiation wavelengths to increase heat onto and/or near the infectious organisms, and/or;
d.) simultaneously or by alternating turns, applying and/or projecting the antimicrobial lighting as a first set of electromagnetic radiation wavelength(s) and the Red and/or IR lighting electromagnetic radiation wavelength(s) as a second set of electromagnetic radiation wavelength(s) that are focused onto and/or near the microbial type and other infections within a living species to reduce and/or kill invading and/or unwanted infections and/or microorganisms on and/or within a living species, individually and/or in combination hereinafter AILR and/or AILR-MD.

[Para 33] According to another aspect of the invention, the antimicrobial lighting devices and/or systems of the invention can be used to kill unwanted parasites, organisms and/or microorganisms and/or infections, hereinafter “infections”, (for example COVID-19, MERSA, Cancer or other infections) infecting a living species, and the Red and/or IR lighting devices and/or systems can be used to increase heat directly onto and/or near the targeted, unwanted infections similar to a fever thereby slowing down the infections ability to multiply and/or infect more healthy cells and/or tissue. The antimicrobial light and/or in conjunction with the IR heat delivered as a targeted, focused and/or localized fever effect would support and/or assist the immune systems white blood cells to better surround the infectious organisms thereby eventually slowing and/or killing off the infection within the living species just as they do when a living species produces a fever.

[Para 34] Using 100-350 nm UV lighting can be more dangerous and challenging than using 350-1400 nm lighting in medical devices and/or applications where energy using these wavelengths on or within living beings and/or species requiring rapid elimination of infectious microbial diseases that are creating risk of damaging and/or loss of limbs, organs and/or life.

[Para 35] According to one aspect of the invention, with proper considerations relating to process, implementation, system design, time/duration and/or energy levels, concentration and/or placement of such energy and other criteria, antimicrobial lighting devices that deliver 380-420 nm, and potentially wavelength ranges of 350-450 nm that are still within the outer edge or just outside of the UV spectrum, and/or devices that deliver Red and/or IR light and/or energy separately and/or simultaneously with the antimicrobial lighting devices, would therefore be much safer to use in medical lighting devices and/or systems designed for eliminating microorganisms and/or infections that are invading living species, organs and/or tissue. Such devices and/or systems could be used in medical treatments for reducing and/or eliminating unwanted microorganisms within living species and/or living tissue without the same negative effects of UV lighting below 350 nm wavelengths.

[Para 36] According to another aspect of the invention, since light wavelengths in the 380 nm - 420 nm range have proven to be effective in killing over 99% of bacteria over time based on intensity of light and specific wavelengths, it is contemplated that placing light internally into a living species organ, or by projecting sufficient levels of light energy and/or intensity needed to pass through living tissue and reach the specific infectious organisms, would effectively and rapidly reduce and/or kill the invading infectious organisms over a shorter period of time compared to not treating the infection with the AILR-MD.

[Para 37] According to another aspect of the invention, with IR light/energy wavelengths in the 700-1400 nm range being proven to increase heat, improve oxygen levels, increase circulation, reduce inflammation and deliver other health benefits, it is contemplated that placing such light and/or wavelength energy(s) internally into a living species organ, or by projecting sufficient levels of light energy needed to pass through living tissue and reach the specific infectious organisms, would aid in effectively and rapidly reducing and/or killing the invading infections over a shorter period of time compared to not treating the infection with light AILR-MD.

[Para 38] According to another aspect of the invention, it is further contemplated that by concentrating and/or projecting such light wavelengths of 350-450 nm and more specifically 380-420 nm, with or without phosphor conversion of such wavelengths, (hereinafter Visible Anti-Infective Lighting or “VAIL”), and/or by concentrating and/or projecting Red 650-720 nm, and more specifically IR light/energy wavelengths of 700-1200 nm (hereinafter Infrared Fever Lighting or “IFL”), and placing, projecting and/or concentrating such light and/or electromagnetic radiation wavelength energy(s) onto and/or internally into a living species organ, or by projecting sufficient levels of such electromagnetic radiation energy(s) needed to pass through living tissue and reach the specific infectious organisms, would effectively and rapidly reduce and/or kill the invading infections over a shorter period of time compared to not treating the infection with AILR-MD.

[Para 39] According to another aspect of the invention, it is contemplated that:
a. one or a combination and/or group of VAIL wavelengths could be used in devices according to the invention, and/or;
b. one or a combination and/or group of IFL wavelengths could be used in devices according to the invention, and/or;
c. one or a combination and/or group of both VAIL and IFL wavelengths could be used in alternating modes and/or simultaneously in separate and individual, or single medical lighting devices and/or systems, in either respect together or separately considered “ALRMD”” according to the invention.

[Para 40] According to another aspect of the invention, VAIL and/or IFL light sources and/or devices could be integrated together and/or combined into a single device to provide an output of both forms and/or categories of antimicrobial light (VAIL) for reducing and killing infectious organisms, and IR wavelength energy(s) (IFL) to reduce inflammation and/or create and/or induce a targeted fever effect on certain cells simultaneously for the purposes of proving ALRMD procedures and devices for killing unwanted infections and/or organisms within a living species. The VAIL and IFL light sources and/or devices could provide one or a combination of a constant output, pulsed output, modulated output, sensor responsive output, time based output or variable output of one or more light and/or wavelengths of radiation energy from one of both VAIL and IFL light sources and/or devices.

[Para 41] According to another aspect of the invention, VAIL and IFL light sources and/or devices could operate on constant voltage, constant current, AC voltage, DC voltage, pulse width modulation “PWM”, battery power, universal voltage input power supplies, inverters, solar power or any other form of power that could power and/or drive electronic circuits and/or lighting devices.

[Para 42] According to another aspect of the invention, ALRMD and/or treatments could be used and/or provided separately, or in conjunction with other medical procedures and/or treatments including but not limited to drug therapy, surgery, sensing, photo imaging, bronchoscopy, ultrasound, measuring, monitoring, oxygen delivery, sonic, nano-medical robots and other procedures. A single device could provide and/or deliver one or a combination of VAIL and/or IFL energy treatment. VAIL and/or IFL devices could be integrated and/or combined with other medical devices and/or non-medical items including but not limited to nano-medical robots, endoscopes, bronchoscope, cameras, ventilators, electrical stimulators, implanted devices, wearable devices, full and/or partial patient enclosures, medical rooms, ceilings, walls, floors, patient beds and/or tables, chairs, prosthetics, ceiling lights, light bulbs, portable devices, communications devices, video displays, handheld devices, and more.

[Para 43] According to another aspect of the invention, one example method of treatment could include but not be limited to a person partially or completely sitting, laying, being covered, wrapped and/or enclosed within a ALRMD procedure device for a period of time for killing unwanted infections and/or organisms within a living species.

[Para 44] According to another aspect of the invention the ALRMD wavelengths could be set and/or tuned at one or more specific selected wavelengths 405 nm and/or 850 nm for example, that fall within the range of 350 nm - 450 nm and/or 700 nm - 1400 nm based on the infection, information, feedback data and/or response of the infectious cells, amount and/or depth of tissue needing to be penetrated, or other factors. The setting, control and/or tuning of the AILMD output wavelengths could be done manually, electronically and/or automatically according to the invention and the setting, control and/or tuning of such wavelengths could be at one or more similar or different levels of output energy levels per output wavelength. Planck’s equation λ = hc/e could be used to calculate the electromagnetic radiation output energy and or to set the desired output wavelength energy(s). An output VAIL wavelength of 405 nm could be provided at 10 watts or 100 lux, while an IFL output wavelength of 850 nm could be provided at 20 watts for example, but not limited to these specific power levels and/or wavelengths. One or more wavelengths and/or output energy levels from the ALRMD could also be set to be delivered in various ways including but not limited to a constant, pulsed, pulse width modulated, modulated or timed and such outputs could be controlled, set and/or programmed by the user of the ALRMD and/or systems.

[Para 45] According to another aspect of the invention, one example method of treatment could include but not be limited to the following: In the case of a respiratory infection such as SARS or COVID-19 were to invade the respiratory track or lungs of a human, or a staphylococcal infection were to invade a diabetic persons leg, or travel to another organ, using one wavelength, or a combination of radiation wavelengths and/or light energy between the ranges of 350-1400 nm could be used to reduce and/or kill microbial infections on and/or within living species. For example, 405 nm of light energy at specific desired and controlled durations of time, power, distribution and/or beam angles, and/or intensity levels could be administered to reduce and/or kill the microbial infection inside the lungs or other parts of the body, or within other living species and/or tissues or organs according to the inventions and methods described herein. Another option would be to use and deliver IR energy somewhere in the ranges of 700 nm - 1 mm in conjunction with such antimicrobial light energy. The IR lighting devices and/or wavelengths can be used to reduce inflammation and/or increase heat directly onto and/or near the targeted, unwanted infections similar to a natural fever response thereby slowing down the infections ability to multiply and/or infect more healthy cells and/or tissue. The antimicrobial light along with the heat/fever delivered as a targeted, focused and/or localized area would support and/or assist the immune systems white blood cells and/or anti-microbial light energy, to better and more successfully fight off the infectious cells thereby eventually slowing and/or killing off the microbial infection within the living species.

[Para 46] According to another aspect of the invention, such treatments and/or devices could include for example but not be limited to, a flexible fiber optic and/or quartz fiber optic type cable having sidewall emission of light along at least a portion of the length of cable, or a bronchoscope having an outer layer that would be illuminated with one or more wavelengths somewhere within the range of 350-450 nm, and more specifically 380-420 nm, could be inserted into the lungs and light up the inside of the lungs with antimicrobial light to reduce and/or kill harmful infectious diseases. Simultaneously or alternatively a light source could be placed inside the living species under the skin and near the exterior walls of an organ such as the lung, or outside of the living species facing into the skin and a specific targeted organ and/or area, and project a sufficient level of wavelength energy needed to penetrate layers of living tissue and reach the microorganisms would effectively and rapidly reduce and/or eliminate unwanted microbial infections.

[Para 47] According to another aspect of the invention, many various forms of lighting devices and/or systems could be designed and produced to be optimized for various medical requirements where antimicrobial lighting devices for eliminating such infections in living species would be used and applied including but not limited to flexible, rigid, flat, linear, tubular, round, rectangular, stranded, flat panels of other structures that can be designed to deliver light at the desired ALR wavelengths.

[Para 48] According to another aspect of the invention, as long as the desired ALR wavelengths and energy levels could be achieved and controlled, and devices could be designed to achieve the desired objective for their applications of use, technologies used in such lighting devices and/or systems for eliminating microbial infections in living species could include but not be limited to LEDs, OLEDs, micro-LEDs, laser diodes, bioluminescent organisms, incandescent, halogen, xenon, mercury vapor, fluorescent or other light producing technologies.

[Para 49] According to another aspect of the invention, devices and/or techniques to deliver light from lighting devices for eliminating such infections in living species could include but not be limited to fiber optics, laser, edge lit and/or light piping, optics, solid state controllable optics, reflectors and more. The antimicrobial light could be delivered in broad distribution covering large areas of infected and/or non-infected living tissue and/or cells, or concentrated with optics to focus the light onto a specific area of infected and/or non-infected tissue and/or cells.

[Para 50] According to another aspect of the invention, placing such ALR on and/or near living tissue and/or cells, where the amount of light radiation is sufficient enough to penetrate through one or more layers of living tissue and reach infections, such threatening infections could effectively be reduced and/or eliminated with and/or without the added support of unproven and/or undesired pharmaceutical drugs that may require more time to test, approve, don’t work, or introduce risk and/or side effects.

[Para 51] According to another aspect of the invention, lighting devices including but not limited to LEDs may or may not use a phosphor to provide a phosphor converted output wavelength and/or color of light from the original output wavelength produced by the lighting device. If white light converted by phosphor is desired, it could be assembled similarly to a “blue-phosphor” LED device which includes a semiconductor LED that emits a majority of light/peak of light within the 380-420 nm wavelength range rather than wavelengths within the conventional range of approximately 450-495 nm, which would be perceived as blue. Light in the 380-420 nm wavelength is capable of killing or deactivating microorganisms such as but not limited to Gram positive bacteria, Gram negative bacteria, bacterial endospores, and yeast and filamentous fungi. Some Gram positive bacteria that can be killed or deactivated include Staphylococcus aureus (incl. MRSA). Clostridium perfringens. Clostridium difficile. Enterococcus faecalis, Staphylococcus epidermidis, Staphyloccocus hyicus, Streptococcus pyogenes, Listeria monocytogenes, Bacillus cereus, and Mycobacterium terrae. Some, Gram negative bacteria include Acinetobacter baumannii, Pseudomonas aeruginosa, Klebsiella pneumoniae, Proteus vulgaris, Escherichia coli, Salmonella enteritidis, Shigella sonnei, and Serratia spp. Some bacterial endospores include Bacillus cereus and Clostridium difficile. Some yeast and filamentous fungi include Aspergillus niger, Candida albicans, and Saccharomyces cerevisiae. Light in the 380-420 nm wavelength has been effective against every type of bacteria tested, although it takes different amounts of time or dosages and/or energy levels dependent on species. Based on known results it is expected to be effective against all gram-negative and gram-positive bacteria to some extent over a period of time. It can also be effective against many varieties of fungi, although these will take longer to show an effect. The LED, according to embodiments of the disclosure, may be surrounded by a phosphor material capable of absorbing and converting some portion of that anti-microbial light emitted from the LED (380-420 nm) to an alternative wavelength or wavelengths. This LED device can have a combination of selected phosphors, such as but not limited to Lutetium Aluminum Garnet and Nitride, that when combined at the proper ratios can emit a light perceived as white or a hue of white. This example LED device can have a CRI equal to or greater than 70. In some embodiments, this example LED device can have a CRI equal to or greater than 80. A percentage of spectral content of light emitted from the example LED device with approximately 380-420 nm wavelength can be equal to or greater than 20%. In some embodiments, light with wavelengths in the range from approximately 380-420 nm may comprise at least approximately 25%, 30%, 35%. 40%, 45%, or 50% of the total combined light emitted from the example LED device.

[Para 52] Another aspect of the invention is to combine at least one LED chip having at least one output wavelength somewhere in the range of 380-420 nm, and at least one IR LED chip having at least one output wavelength somewhere in the range of 700 nm - 1 mm into a single blue/IR LED package “BIR” LED package. The BIR LED package may include input and output and/or positive and negative “+/-” electrical connections to deliver a voltage and/or current to both of the LED chips either at independent times or at the same time, or alternately may have separate positive and negative electrical connections to the blue LED chip(s) and IR LED chip(s) allowing for different voltage and/or current levels to be delivered to the blue and IR LEDs chips in the single package.

Another aspect of the invention is to combine at least one 380-420 nm blue LED chip and at least one 700 nm ― 1 mm IR LED chip into a single blue/IR LED package “BIR” LED package. The BIR LED package may include input and output and/or positive and negative “+/-” electrical connections to deliver a voltage and/or current to both of the LED chips at the same time, or alternately may have separate positive and negative electrical connections to each of the blue LED chip(s) sections and IR LED chip(s) sections allowing for different voltage and/or current levels to be delivered to the blue and IR LEDs chips in the single package. When more than one blue LED chip(s) is packaged and/or more than one IR LED chip(s) is packaged in a single package, the blue may be one or more different wavelengths (405 nm and 410 nm for example), and the IR LED chips may be one or more different wavelengths (750 nm, 800 nm and 850 nm for example). In addition to having the option of delivering different voltage and/or current levels to the different LED chips, different drive methods could be used for a single package. For example, the blue LED chips could be powered with a constant voltage or constant current, while the IP LED chips in the same package could be powered with the same/or different voltage or current level, but be pulsed o and off, or be pulsed at higher currents for a given period of time. Various drivers and/or power supplies as well as drive schemes could be used to drive such LED packages including but not limited to constant voltage, constant current, PWM, high frequency AC, high voltage AC or high voltage rectified AC, linear step drive, buck boost, or other LED driver and/or methods known to those skilled in the art. One or more of the blue LED chips inside the BIR package may or may not be surrounded and/or coated with a phosphor and more than one BIR chips and/or packages may be integrated into a single assembly and/or substate. The assembly and/or substrate may be made of various material including but not limited to printed circuit board “PCB, metal core PCB “MCPCB”, GaN, Sapphire, Silicon, aluminum, metal, glass, copper or other metals. Additionally, these and/or other material may be used individually or in combination for heat sinking the BIR LED packages, assemblies and or AILR devices and systems.

[Para 53] Another aspect of the invention is to combine at least one LED package having at least one 380 nm - 420 nm blue LED chip(s), and at least one LED package having at least one 700 nm - 1 mm IR LED chip(s) onto separate substrates and/or printed circuit boards “PCBs” or a single substrate and/or PCB with such separate and/or or single substrates being capable of being integrated into separate and/or a single lighting device and/or system assembly thereby providing a Blue/IR Assembly or “BIR assembly”. The BIR assembly may include input and output and/or positive and negative “+/-” electrical connections to deliver voltage and/or current to both blue and IR wavelength options at the same time, or alternately may have separate positive and negative electrical connections individually to one or more of the blue LED package(s) and IR LED package(s) allowing for different voltage and/or current levels to be delivered to the blue and IR LED chips and/or packages on the BIR assembly(s). When more than one blue LED package and/or more than one IR LED package is placed on a substrate, the blue may be one or more different wavelengths (405 nm and 410 nm for example), and the IR LED chips may be one or more different wavelengths (750 nm, 800 nm and 850 nm for example). In addition to having the option of delivering different voltage and/or current levels to the different LED chips, different drive methods could be used for a single package. For example, the blue LED chips could be powered with a constant voltage or constant current, while the IP LED chips in the same package could be powered with the same/or different voltage or current level, but be pulsed o and off, or be pulsed at higher currents for a given period of time. Various drivers and/or power supplies as well as drive schemes could be used to drive such LED packages including but not limited to constant voltage, constant current, PWM, high frequency AC, high voltage AC or high voltage rectified AC, linear step drive, buck boost, or other LED driver and/or methods known to those skilled in the art.

[Para 54] Other advantages and aspects of the present invention will become apparent upon reading the following description of the drawings and detailed description of the invention.

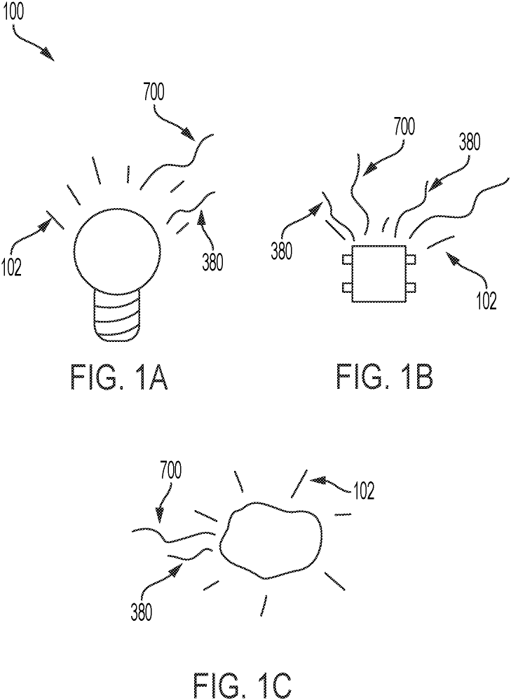

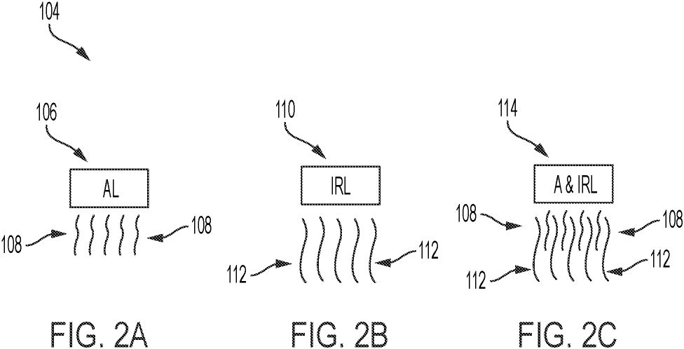

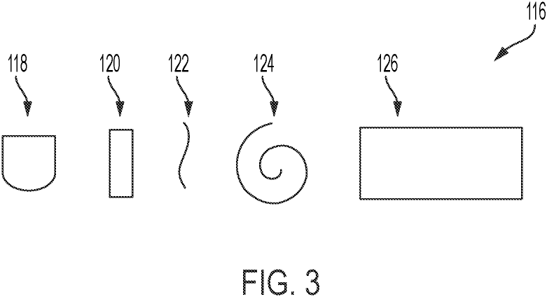

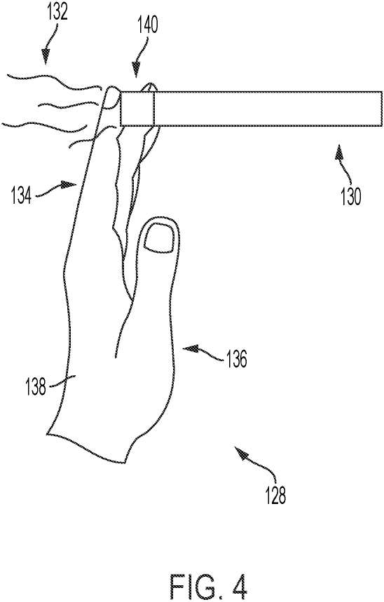

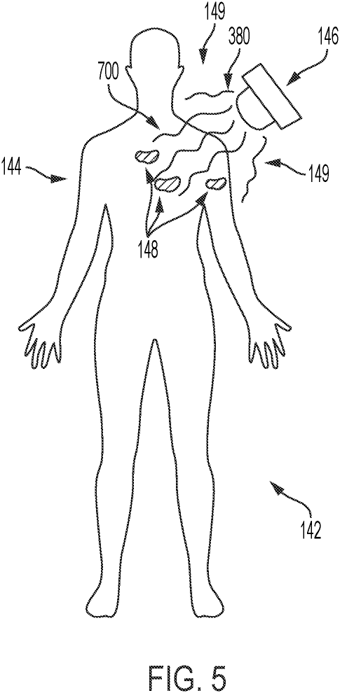

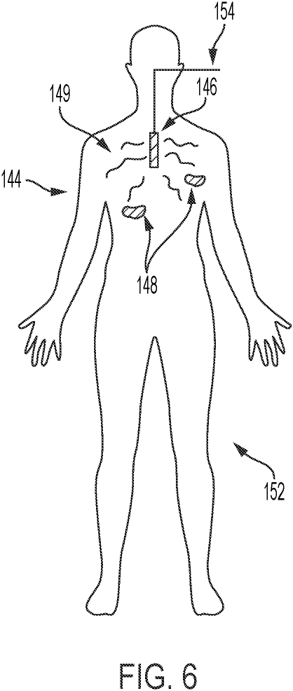

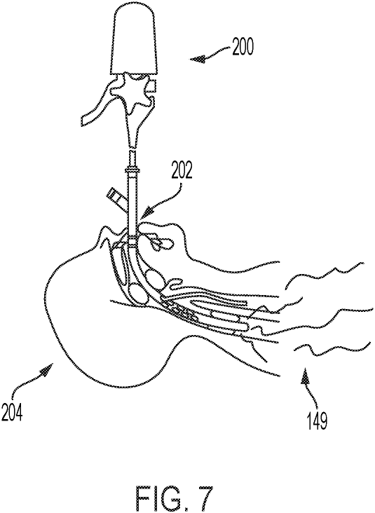

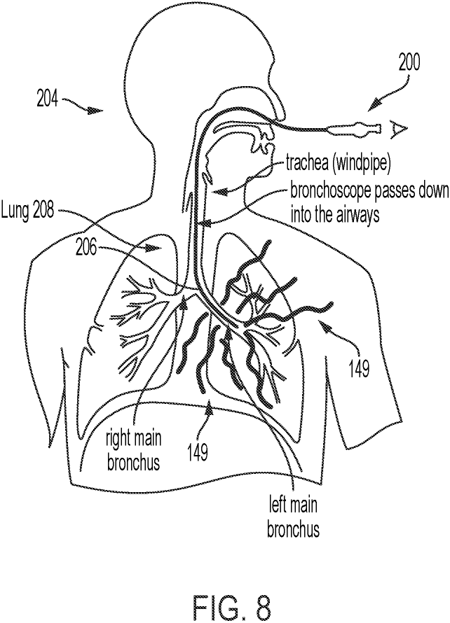

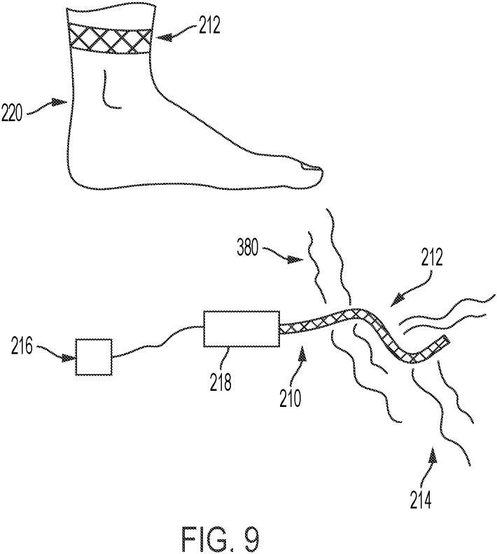

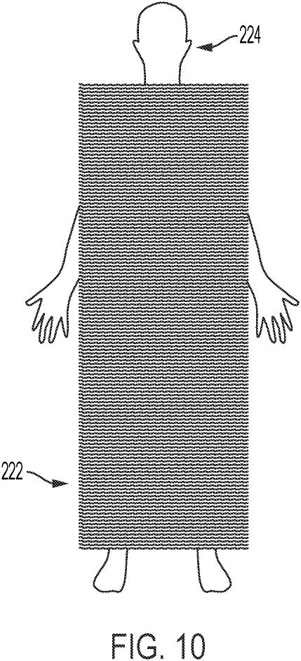

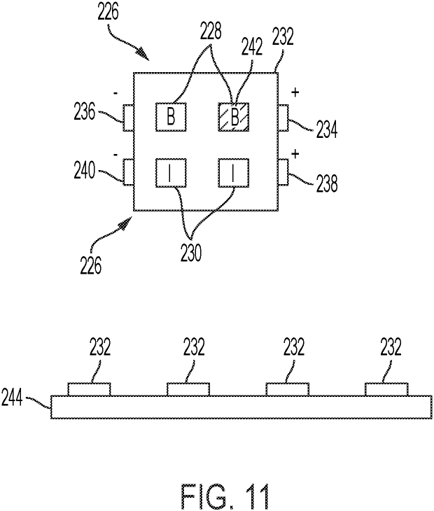

### BRIEF DESCRIPTION OF THE DRAWINGS

[Para 55] FIG. 1 shows a schematic view of a preferred embodiment according to invention;

[Para 56] FIG. 2 shows a schematic view of a preferred embodiment according to invention;

[Para 57] FIG. 3 shows a schematic view of a preferred embodiment according to invention;

[Para 58] FIG. 4 shows a schematic view of a preferred embodiment according to invention;

[Para 59] FIG. 5 shows a schematic view of a preferred embodiment according to invention;

[Para 60] FIG. 6 shows a schematic view of a preferred embodiment according to invention;

[Para 61] FIG. 7 shows a schematic view of a preferred embodiment according to invention;

[Para 62] FIG. 8 shows a schematic view of a preferred embodiment according to invention;

[Para 63] FIG. 9 shows a schematic view of a preferred embodiment according to invention;

[Para 64] FIG. 10 shows a schematic view of a preferred embodiment according to invention; and

[Para 65] FIG. 11 shows a schematic view of a preferred embodiment according to invention.

### DETAILED DESCRIPTION

[Para 66] While this invention is susceptible to embodiments in many different forms, there is described in detail herein, various embodiments of the invention with the understanding that the present disclosures are to be considered as exemplifications of the principles of the invention and are not intended to limit the broad aspects of the invention to the embodiments illustrated.

[Para 67] The present invention is directed to multiple anti-infective lighting methods, devices and/or systems for eliminating infections in living species. The lighting devices and/or systems may or may not be integrated with other devices. As discussed herein, a lighting device may include any device capable of emitting light no matter the intention. Examples of lighting devices which are contemplated by this invention include, but are not limited to LEDs, OLEDs, micro-LEDs, laser diodes, incandescent, halogen, xenon, mercury vapor, fluorescent or other light producing devices, and can potentially one day include bioluminescent living species, organisms and/or cells that could be engineered, genetically modified and_{/}or developed to support the technologies and methods that produce the wavelength energy(s) and used in ways according to the inventions described herein. The devices and/or systems may also include one or more of power connections or leads, contacts, drivers, transistors, resistors, capacitors, inductors, diodes, integrated circuits “IC”s, antennas, fuses, sensors, feedback, firmware, software, or other devices required to provide, control and/or manage power to circuits and device in order to emit the AIRL. A lighting system may include multiple such devices, and some or all of the required parts to drive such a device or multiple devices, including but not limited to, power supplies, transformers, inverters, rectifiers, sensors or light emitting circuitry discussed herein. While a lighting device according to the invention may be incorporated into one or more of a lighting system, a lamp, a light bulb, a room, medical devices and/or non-medical devices/items including but not limited to nano-medical robots, endoscopes, bronchoscope, cameras, ventilators, electrical stimulators, implanted devices, wearable devices, full and/or partial patient enclosures, medical rooms, ceilings, walls, floors, patient beds and/or tables, chairs, prosthetics, ceiling lights, portable devices, communications devices, video displays, handheld devices, and more.

[Para 68] The purposes of the devices described herein are multi-fold and may be accomplished independent of each other. One intention of the methods and devices described herein is to provide anti-infective and/or antimicrobial light near and/or directly onto infectious living cells on and/or within a living species. Another intention of the methods and devices described herein is to provide IR light and/or energy(s) directly near and/or onto infectious living cells on and/or within a living species. Another intention of the methods and devices described herein is to provide antimicrobial light and IR light near and/or directly onto living cells on and/or within a living species. Another intention of the methods and devices described herein is integrated such light delivery devices into lighting systems and/or together with and/or in other devices and/or items as described in some examples herein.

[Para 69] In order to achieve any of the goals of the devices described herein, it may be necessary to include one or more additional medical processes and/or procedures prior to, after and/or in conjunction with the methods and/or devices according to the invention.

[Para 70] FIG. 1 shows example light sources 100 according to the invention with such example light sources including but not limited to a light bulb and/or lamp 1.A, at least one an LED 1.B, and an organic light source 1.C. the example light sources would have the ability to provide an output of at least one or more wavelengths 102 of one or a combination of VAICL wavelengths 380 and ICTFL wavelengths 700 (anti-infective light radiation) needed to effectively provide Anti-Infective Lighting Methods & Devices and/or “AILMD” according to the invention.

[Para 71] FIG. 2 shows example lighting devices and/or systems 104 according to the invention. The example lighting devices and/or system in FIG. 2.a, FIG. 2.b, and FIG. 2.c provide one or a combination of output wavelengths. FIG. 2.a shows a VAICL lighting device 106 that produces at least one VAICL output wavelength(s) 108 according to the invention. FIG. 2.b. shows an ICTFL lighting device 110 that produces at least one ICTFL output wavelength(s) 112 according to the invention. FIG. 2.c shows a lighting device 114 that produces a combination of one of more VAICL output wavelength(s) 108 and ICTFL output wavelengths 112 according to the invention. One or more of any of the lighting devices and/or systems 106, 110 and/or 114, one or a combination of being an example of AILMD, may be used for eliminating infections on the exterior and/or interior of living species including but not limited to humans, animals, mammals and other living species by:
providing lighting devices, that from the exterior of a living species and/or when integrated or placed within the interior of a living species, will project sufficient levels of light and/or IR energy directly onto and/or through one or more layers of living tissue so that the light and/or IR energy reaches microbial infections, and;
using antimicrobial lighting devices that produce one or a combination and/or group of wavelengths in the range of 350-450 nm, and more specifically 380-420 nm, and/or using Red and/or infrared radiation (IR) lighting and/or devices that produce one or a combination and/or group of wavelengths in the range of 625-1200 nm, and;
individually using the antimicrobial lighting and/or wavelengths to increase heat onto and/or near the microbial infections, and/or;
simultaneously applying and/or projecting the antimicrobial lighting as a first set of electromagnetic energy wavelength(s) and the Red and/or IR lighting and/or wavelength(s) as a second set of electromagnetic energy wavelength(s) that are focused onto and/or near the microbial infections within a living species to reduce and/or kill invading and/or unwanted infections and/or microorganisms on and/or within a living species.

[Para 72] FIG. 3 shows various example AILMD lighting devices and/or systems 116 according to the invention. The AILMD devices and/or systems 118, 120, 122, 124 and 126 shown depict how the various example AILMD devices and/or systems 116 may be made in different shapes and sizes or various materials including but not limited to flexible, rigid, flat, linear, tubular, completely or partially round, rectangular, stranded, flat panels, metal, plastic, silicone, organic material, biodegradable material and/or other shapes, sizes and structures that can be designed as needed to deliver light at the desired wavelengths according to the design requirements of the AILMD devices and/or systems.

[Para 73] FIG. 4 shows an example image 128 of how VAICL, ICTFL and/or AILMD lighting devices and/or systems could work on a living species according to the invention. At certain levels of power and/or brightness, electromagnetic energy wavelengths (anti-infective lighting) can pass through living species and/or living species tissue. Many living species and/or one or more layers of living species tissue can be translucent. In this example a flashlight 130 is shown projecting waveforms 132 of light through a living species and/or finger 134 of a human hand 136. It is known that if you take a light sources such as a flashlight 130 and press firmly enough into a finger 134 or other areas of living tissue 138 on a living species while pointing the output wavelengths 132 of light into one side of a finger 134 or other living tissue 138, the wavelengths 132 of light energy will pass through one or more layers of the finger 134 and/or other living tissue 138.

[Para 74] FIG. 5 shows an example image 142 of human and/or living species 144 and an AILMD lighting device and/or system 146 being placed and operating from the exterior of the living species 144 to reduce and/or eliminate unwanted infectious organisms 148 on and/or within the living species 144 by radiating one or more wavelengths 149 onto, into and/or through the tissue of the living species, according to an embodiment of the present disclosure. The AILMD lighting device and/or system 146 may provide one of more output wavelengths of energy(s) of at least one or more wavelengths 149 of one or a combination of VAICL wavelengths 380 and/or ICTFL wavelengths 700 needed to effectively provide Anti-Infective Lighting Methods & Devices and/or “AILMD” 146 according to the invention. By providing sufficient levels of output wavelength energy, the wavelengths 149 would be delivered directly onto and/or through one or more layers of living tissue so that the electromagnetic wavelength energy(s) reach near or directly onto unwanted infectious living cells similar to radiation therapy when used on cancer yet substantially safer for living cells surrounding the infectious cells 148. It is contemplated that the AILMD wavelengths 149 could be set and/or tuned at one or more specific selected wavelengths 380 and/or 700 for example, that fall within the range of 350 nm - 450 nm and/or 700 nm - 1400 nm based on the infection, information, feedback data and/or response of the infectious cells, amount and/or depth of tissue needing to be penetrated, or other factors. The tuning of the AILMD output wavelengths 149 could be done manually and/or automatically according to the invention and the setting and/or tuning of such wavelengths 149 could be at one or more similar or different levels of output energy levels per output wavelength. One or more wavelengths could also be set to be delivered and/or output energy from the AILMD devices in various ways including but not limited to a constant, pulsed, pulse width modulated, modulated, timed and that such outputs could be controlled, set and/or programmed by the user of the AILMD devices and/or systems 146.

[Para 75] FIG. 6 shows an example image 152 of human and/or living species 144 and an AILMD lighting device and/or system 146 being placed and operating from the interior of the living species 144 to reduce and/or eliminate unwanted infectious organisms 148 on and/or within the living species 144 by radiating one or more wavelengths 149 onto, into and/or through the tissue of the living species, according to an embodiment of the present disclosure. The AILMD lighting device and/or system 146 may provide one of more output wavelengths of energy(s) of at least one or more wavelengths 149 of one or as described in FIG. 5, a combination of VAICL wavelengths 380 and/or ICTFL wavelengths 700 needed to effectively provide Anti-Infective Lighting Methods & Devices and/or “AILMD” 146 according to the invention. By providing sufficient levels of output wavelength energy, the wavelengths 149 would be delivered directly onto and/or through one or more layers of living tissue so that the electromagnetic wavelength energy(s) reach near or directly onto unwanted infectious living cells similar to existing radiation therapies used in cancer treatment however using electromagnetic radiation in the visible spectrum of wavelengths in the range of 380-450 nm and/or IR wavelengths in the range of 700-1200 nm is substantially different and safer for living species and or living cells surrounding the infectious cells 148 one would wish to eliminate. It is contemplated that the AILMD wavelengths 149 could be set and/or tuned at one or more specific selected wavelengths 380 and/or 700 for example as described in FIG. 5 that fall within the range of 350 nm - 450 nm and/or 700 nm - 1400 nm based on the infection, information, feedback data and/or response of the infectious cells, amount and/or depth of tissue needing to be penetrated, or other factors. The tuning of the AILMD output wavelengths 149 could be done manually and/or automatically according to the invention and the setting and/or tuning of such wavelengths 149 could be at one or more similar or different levels of output energy levels per output wavelength. One or more wavelengths could also be set to be delivered and/or output energy from the AILMD devices in various ways including but not limited to a constant, pulsed, pulse width modulated, modulated, timed and that such outputs could be controlled, set and/or programmed by the user of the AILMD devices and/or systems 146. The AILMD devices and/or systems 146 could include and/or be connected to at least one or a combination of a wire, hose, tube, fiber optic cable and/or antenna for example, such examples collectively shown in 154 and 154 could be accessible from the interior and/or exterior of the living species 144.

[Para 76] FIG. 7 shows an example AILMD lighting device and/or system 200 inserted through and into the mouth of a living species 204, according to an embodiment of the present disclosure. The AILMD device 200 can have a light source 100 as described in FIG. 1 as part of AILMD device 200. The AILMD device 200 may be integrated with other devices including but not limited to a bronchoscope, a respirator other devices. The devices could include a light emitting section and/or material 204 that emits and/or radiates one or a combination of wavelengths 149 inside a living species as described above in FIG. 6.

[Para 77] FIG. 8 shows an example AILMD lighting device and/or system 200 inserted through and/or into a living species 204 according to the invention. The AILMD device 200 includes a light emitting section and/or material 206 that is placed near and/or into the lungs and emits and/or radiates one or a combination of wavelengths 149 inside a living species as described above in FIG. 6. In this example, a device such as a bronchoscope could include the ability to deliver AILMD wavelength 149 radiation directly inside of a living species lungs 208 that may be infected with a life threatening infectious disease such as Influenza, Covid-19 or other infectious diseases that could be reduced and/or killed using the AILMD devices and/or systems as described herein according to the invention.

[Para 78] FIG. 9 shows one example embodiment image of an AILMD lighting device and/or system 210 wherein the AILMD device 210 may have a flexible section 212 that provides output of one of more wavelengths of energy(s) of at least one or more wavelengths 214 of one or a combination of VAICL wavelengths 380 and/or ICTFL wavelengths 700 needed to effectively provide Anti-Infective Lighting Radiation Methods & Devices and/or “AILRMD” 210 according to the invention. The AILRMD device 210 may have a remote power supply and/or source 216 or an integral power supply and/or source 218. The power supply and/or source may be any form of power supply and/or source that can power electronic devices. The AILRMD device may be placed on and/or wrapped directly onto a body part such as a limb 220 of a human and/or living species to deliver anti-infective light radiation near and/ or directly onto the infectious organisms which can be delivered onto and/or through one or more layers of living tissue so that the electromagnetic wavelength energy(s) reaches near or directly onto unwanted infectious living cells. It is also contemplated that many other types of wearables can be designed as AIRLMD devices and/or systems including but not limited to hats, helmets, wraps and/or pads, vests jackets and/or boots.

[Para 79] FIG. 10 shows one example embodiment image of an AILRMD lighting device and/or system 222 wherein a living species 224 may be completely or partially covered and/or enclosed within an AILMD device 222 and receive treatments using Anti-Infective Lighting Radiation Methods & Devices and/or “AILRMD” according to the invention.

[Para 80] FIG. 11 shows one example embodiment image of a Anti-Infective Lighting Device “AILD” 226 for use in AILRMD devices and/or systems as described above in previous figures according to the invention. In this example, the AILD 226 is combines at least one 380-420 nm blue LED chip 228 (as an optional light source technology) and at least one 700 nm - 1 mm IR LED chip 230 into a single blue/IR LED package “BIR” LED package 232. The BIR LED package 232 may include input and output and/or positive 234 and negative 236 “+/” electrical connections to deliver voltage and/or current to both of the LED chips at the same time, or alternately may have separate positive 238 and negative 240 electrical connections individually to each of the blue LED chip(s) 228 and IR LED chip(s) 230 allowing for different voltage and/or current levels to be delivered to the blue and IR LEDs chips in the single package. The LED chips may be connected in series, parallel and/or series/parallel within the BIR LED package 232. When more than one blue LED chip 228 is packaged and/or more than one IR LED chip 230 is packaged in a single BIR LED package, the blue output wavelengths may be one or more different wavelengths (405 nm and 410 nm for example), and the IR LED chips may be one or more different wavelengths (750 nm, 800 nm and 850 nm for example). In addition to having the option of delivering different voltage and/or current levels to the different LED chips, different drive methods could be used for a single package. For example, the blue LED chips 228 could be powered with a constant voltage or constant current, while the IR LED chips 230 in the same package could be powered with the same/or different voltage or current level, be pulsed on and off, or be pulsed at higher currents for a given period of time compared to the blue. Various drivers and/or power supplies as well as drive schemes could be used to drive such BIR LED packages including but not limited to constant voltage, constant current, PWM, high frequency AC, high voltage AC or high voltage rectified AC, linear step drive, buck boost, or other LED driver and/or methods known to those skilled in the art. One or more of the blue LED chips 228 inside the BIR LED package 232 may or may not be surrounded and/or coated with a phosphor 242 and more than one BIR LED package 232 may be integrated into a single assembly 244 which may be a printed circuit board “PCB” material or other substrate and/or receptacle that can house the specific light source technology being used to create the AILRMD devices and/or systems.

[Para 81] Such example current limiting or current controlled diode (CLD). Both CCRs and CLDs actively limit the current flowing through a particular circuit or device by substantially limiting the current to, and maintaining the current at, a threshold level once the current in a connected circuit or device has reached or exceeded a particular value. Using such devices is advantageous over using current limiting resistors insofar as CCRs and CLDs both cap the total current which is allowed to flow through a connected circuit or device, while the resistor only acts to reduce any every climbing current. With a current limiting resistor, as the input voltage to the circuit continues to increase, the current will likewise continue to increase without limit, albeit it at a lower value than without the resistor. With a CCR or a CLD, once the current reaches a threshold maximum, the current will remain substantially constant until the input voltage is reduced, even if the input voltage continues to climb. As will be described herein, in some cases the combination of a CCR or CLD and a current limiting resistor may be beneficial or required.

[Para 82] While both CCRs and CLDs may be used interchangeably to accomplish the goals of the devices described herein, there are differences between the devices. The primary difference between the devices is that CCRs, like those sold by ON Semiconductor, typically have internal transistor based control circuits and have little or no turn on voltage. CLDs are a form of a diode which are based in part on a JFET having a gate shorted to the power source and have a measurable turn on voltage. While the CLDs may be utilized with any of the devices described herein, it may be advantageous to use a CCR when possible in order to avoid the additional turn on voltage requirements of the CLD. However, CCRs and CLDs may be used interchangeably to accomplish the goals of the invention.

[Para 83] FIGS. 2-5 show exemplary LED lighting devices capable of emitting color temperature controlled light. As seen in FIG. 2A, lighting device 10 includes at least two LED circuits 12, 14 which are connected in parallel. Each LED circuit 12, 14 includes one or more LEDs 16, 18 respectively. Each LED circuit 12, 14 has a different forward operating voltage and is capable of emitting light having one or more of a different color or a different wavelength than the other circuit. For example, LED circuit 12 may emit amber or yellow light, while LED circuit 14 emits white or blue light. In order to limit the current within either LED circuit 12, 14, an active current limiting device such as a CCR or CLD, shown as CCR 20 connected in series with at least one LED 16 in first circuit 12, may be provided. As seen in FIG. 2B, additional active current limiting devices, like for example CCR 22, may be added to the device so that each LED circuit is connected in series with an active current limiting device. LED device 10 may further include connection leads 24, 26 for connecting the device to an AC power source, like for example mains power or a switch or dimmer connected to mains power. In order to fully utilize AC power and produce a substantially constant light output, device 10 and/or circuits 12, 14 should be configured such that each circuit 12, 14 is capable of emitting light during both a positive and negative phase of the provided AC voltage.

[Para 84] While the foregoing there has set forth embodiments of the invention, it is to be understood that the present invention may be embodied in other forms without departing from the spirit or central characteristics thereof. The present embodiments, therefore, are to be considered in all respects as illustrative and not restrictive, and the invention is not to be limited to the details given herein. While specific embodiments have been illustrated and described, numerous modifications come to mind without significantly departing from the characteristics of the invention and the scope of protection is only limited by the scope of the accompanying claims.

1. An anti-infective radiation device comprising:
at least one lighting device configured to provide an output energy of at least one antimicrobial electromagnetic radiation wavelength(s) within a range of 350 nm - 450 nm,
wherein the at least one lighting device is configured to be directed towards an exterior of a living species and/or integrated and/or placed within an interior of a living species, wherein the at least one lighting device projects one or more levels of the electromagnetic wavelength radiation directly onto and/or through one or more layers of living species tissue so that the electromagnetic wavelength radiation reaches near or directly onto unwanted infectious living cells or organisms, and
wherein the antimicrobial electromagnetic radiation damages or kills unwanted infectious cells or organisms on or within the living species.

2. The anti-infective radiation device of claim 1, wherein the at least one lighting device is integrated into a medical device.

3. The anti-infective radiation device of claim 1, wherein the at least one lighting device is integrated into a wearable medical device.

4. The anti-infective radiation device of claim 1, further comprising at least one LED.

5. The anti-infective radiation device of claim 1, wherein the output energy of at least one antimicrobial electromagnetic radiation wavelength(s) is configured to be set by a user or operator of the anti-infective radiation device.

6. The anti-infective radiation device of claim 1, wherein the output energy of at least one antimicrobial electromagnetic radiation wavelength(s) is configured to be focused or concentrated on a specific infected area of the living species.

7. An anti-infective radiation device comprising:
at least one lighting device configured to provide an output energy of at least one IR electromagnetic radiation wavelengths within a range of 700 nm - 1200 nm,
wherein the at least one lighting device is configured to be directed towards an exterior of a living species and/or integrated and/or placed within an interior of a living species, wherein the at least one lighting device projects one or more levels of electromagnetic radiation wavelength(s) directly onto and/or through one or more layers of the living species tissue so that the electromagnetic radiation wavelength(s) reach near or directly onto unwanted infectious living cells or organisms, and
wherein the IR electromagnetic radiation increases a temperature of infectious cells or organisms on or within the living species.

8. The anti-infective radiation device of claim 7, wherein the at least one lighting device is integrated into a medical device.

9. The anti-infective radiation device of claim 7, wherein the at least one lighting device is integrated into a wearable medical device.

10. The anti-infective radiation device of claim 7, further comprising at least one LED.

11. The anti-infective radiation device of claim 7, wherein the output energy of at least one IR electromagnetic radiation wavelength(s) is configured to be set by a user or operator of the anti-infective radiation device.

12. The anti-infective radiation device of claim 7, wherein the output energy of at least one IR electromagnetic radiation wavelength(s) is configured to be focused or concentrated on a specific infected area of the living species.

13. An anti-infective radiation device comprising:
at least one lighting device configured to provide a first output energy of at least one antimicrobial first electromagnetic radiation wavelength(s) within a range of 100 nm - 450 nm and a second output energy of at least one IR second electromagnetic radiation wavelength(s) within a range of 700 nm - 1200 nm,
wherein the at least one lighting device is configured to be directed towards an exterior of a living species and/or integrated and/or placed within an interior of a living species, wherein the at least one lighting device is configured to project at least one of the first or second electromagnetic radiation wavelength(s) directly onto and/or through one or more layers of living species tissue so that the electromagnetic radiation wavelength(s) reach near or directly onto unwanted infectious living cells or organisms on or within the living species,
wherein the antimicrobial first electromagnetic radiation damages or kills unwanted infectious cells or organisms on or within the living species, and
wherein the IR second electromagnetic radiation increases a temperature of the infectious cells or organisms on or within the living species.

14. The anti-infective radiation device of claim 13, wherein the at least one lighting device is integrated into a medical device.

15. The anti-infective radiation device of claim 13, wherein the at least one lighting device is integrated into a wearable medical device.

16. The anti-infective radiation device of claim 13, further comprising at least one LED.

17. The anti-infective radiation device of claim 13, wherein the output energy of at least one of the antimicrobial first electromagnetic radiation wavelength(s) or the IR second electromagnetic radiation wavelength(s) is configured to be set by a user or operator of the anti-infective radiation device.

18. The anti-infective radiation device of claim 13, wherein the output energy of at least one of the antimicrobial first electromagnetic radiation wavelength(s) or the IR second electromagnetic radiation wavelength(s) is configured to be focused or concentrated on a specific infected area of the living species.

19. The anti-infective radiation device of claim 13, wherein the output energy of at least one of the first antimicrobial electromagnetic radiation wavelength(s) or the IR second electromagnetic radiation wavelength(s) is configured to be pulsed.

20. The anti-infective radiation device of claim 13, wherein the output energy of at least one of the antimicrobial first electromagnetic radiation wavelength(s) or the IR second electromagnetic radiation wavelength(s) is configured to be used with a drug therapy.

21. A device for treating infections comprising:
at least one lighting device positioned at an exterior of a living species and/or integrated or placed within an interior of a living species such that the at least one lighting device projects a level of a combination of at least first and second sets of electromagnetic energy wavelengths directly onto and/or through one or more layers of living species tissue so that the combination of at least the first and second sets of electromagnetic energy wavelengths reach near or directly onto unwanted infectious living organisms,
wherein the first set of electromagnetic energy provides an output of at least one wavelength energy within a range of 100 nm - 450 nm, and
wherein the second set of electromagnetic energy provides an output of at least one wavelength energy within in a range of 700 nm - 1200 nm.

22. A device for treating infections comprising:
at least one lighting device positioned near an exterior of a living species and/or integrated or placed within an interior of a living species such that the at least one lighting device projects a combination of at least first and second sets of electromagnetic energy wavelengths directly onto and/or through one or more layers of living tissue so that the combination of at least the first and second sets of electromagnetic energy wavelengths reach near or directly onto unwanted infectious living organisms,
wherein the first set of electromagnetic energy provides an output of at least one wavelengths energy(s) within a range of 350 nm - 450 nm,
wherein the second set of electromagnetic energy provides an output of at least one wavelengths energy(s) within in a range of 700 nm - 1200 nm, and
wherein the combination of at least the first and second sets of electromagnetic energy(s) reaching near or directly onto the unwanted infectious living cells increases heat directly onto and/or near the unwanted infectious living cells and kills the unwanted infectious living cells within the living species.

23. A device for treating infections comprising:
at least one lighting device, that from an exterior of a living species and/or when integrated or placed within an interior of a living species, configured to project antimicrobial first and/or IR second electromagnetic energy directly onto and/or through one or more layers of living tissue so that the antimicrobial and/or IR electromagnetic energy reaches microbial infections,
wherein the at least one lighting devices provides an output of one or a combination of wavelength energy(s) in a range of 100 nm - 1200 nm,
wherein the at least one lighting device is configured to simultaneously or alternately project the first and second electromagnetic energy wavelengths, and
wherein the second electromagnetic energy wavelength(s) include at least one IR wavelength(s) in a range of 700 nm to 1200 nm and the second electromagnetic energy wavelength includes at least one antimicrobial energy wavelength(s) in a range of 100 nm - 450 nm.

24. (canceled)
